(19) United States
(12) Patent Application Publication
(10) Pub. No.: US 2014/0324181 A1
(43) Pub. Date: Oct. 30, 2014

(54) Patient-Matched Acetabular Component Alignment Device and Method

(71) Applicants: Alisha W Bergin, Southaven, MS (US); Nathaniel M Quinn, Arlington, TN (US); Jackie G Carter-Horne, Memphis, TN (US)

(72) Inventors: Alisha W Bergin, Southaven, MS (US); Nathaniel M Quinn, Arlington, TN (US); Jackie G Carter-Horne, Memphis, TN (US)

(73) Assignee: SMITH & NEPHEW, INC., Memphis, TN (US)

(21) Appl. No.: 14/260,379

(22) Filed: Apr. 24, 2014

Related U.S. Application Data
(60) Provisional application No. 61/816,213, filed on Apr. 26, 2013.

(51) Int. Cl. A61F 2/46; G06F 17/50

(52) U.S. Cl. CPC A61F 2/4609; G06F 17/50
USPC 623/22.12; 606/91; 700/98

(57) ABSTRACT

Embodiments of the invention are directed to a prosthetic hip system that includes acetabular alignment structures and methods. Acetabular alignment structures and methods may include use of patient-matched components configured to reduce tissue disruption during implantation.

### CROSS-REFERENCE TO RELATED APPLICATIONS

This application claims the benefit of U.S. Provisional Application No. 61/816,213, filed Apr. 26, 2013. The disclosure of this prior application is incorporated by reference in its entirety.

### BRIEF DESCRIPTION OF THE DRAWINGS

The accompanying drawings are incorporated in and form a part of the specification, illustrate the embodiments of the invention, and together with the written description serve to explain the principles, characteristics, and features of the invention. In the drawings:

FIG. 1 is a perspective view of a portion of a pelvis.

FIG. 2 is a cross-sectional view of the pelvis of FIG. 1 taken through at least a portion of the ischium (left side of FIG. 2) of the pelvis.

FIG. 3 is a perspective view of portions of a prosthetic hip system being directed toward a pelvis.

FIG. 4 is a cross-sectional view of an embodiment of the prosthetic hip system of FIG. 3 having an insertion guide engaged with the ischium of the pelvis of FIG. 1.

FIG. 5 is a cross-sectional view of the prosthetic hip system of FIG. 4 with a portion of the prosthetic hip system translated toward the acetabulum of the illustrated pelvis.

FIG. 6 is a cross-sectional view of a prosthetic hip system with a fixation guide engaged with the ischium of the pelvis of FIG. 1.

FIG. 7 is a cross-sectional view of the pelvis of FIG. 1 taken through at least a portion of the inferior column (right side of FIG. 7) of the pelvis.

FIG. 8 is a cross-sectional view of an embodiment of a prosthetic hip system engaged with the inferior column of the pelvis of FIG. 7.

FIG. 9 is a cross-sectional view of the pelvis of FIG. 1 taken through at least a portion of the pubis (right side of FIG. 9) of the pelvis.

FIG. 10 is a cross-sectional view of an embodiment of a prosthetic hip system engaged with the pubis of the pelvis of FIG. 9.

### DETAILED DESCRIPTION OF THE EMBODIMENTS

The following descriptions of the depicted embodiments are merely exemplary in nature and are in no way intended to limit the invention, its application, or uses.

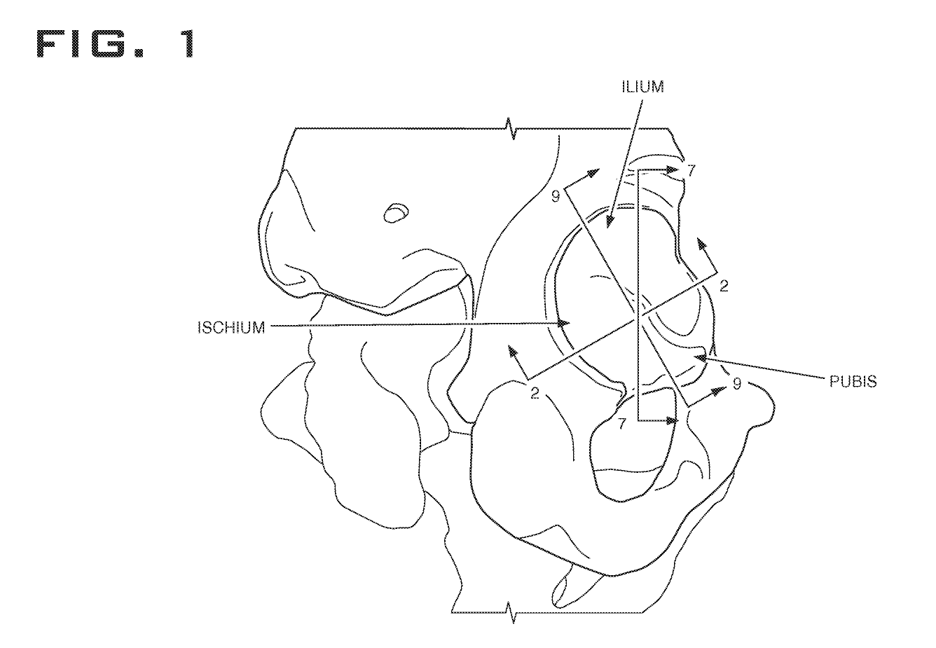

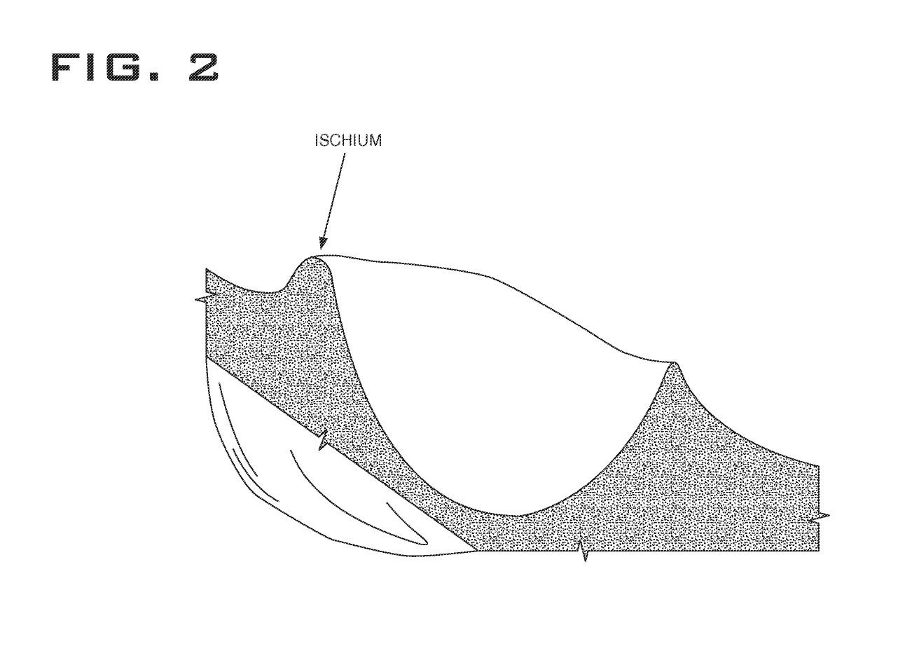

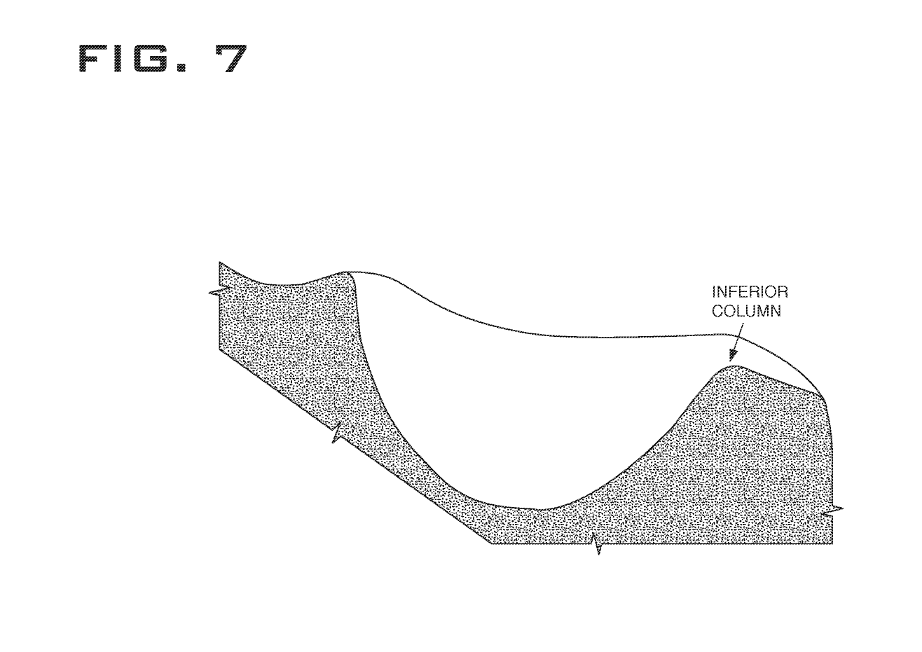

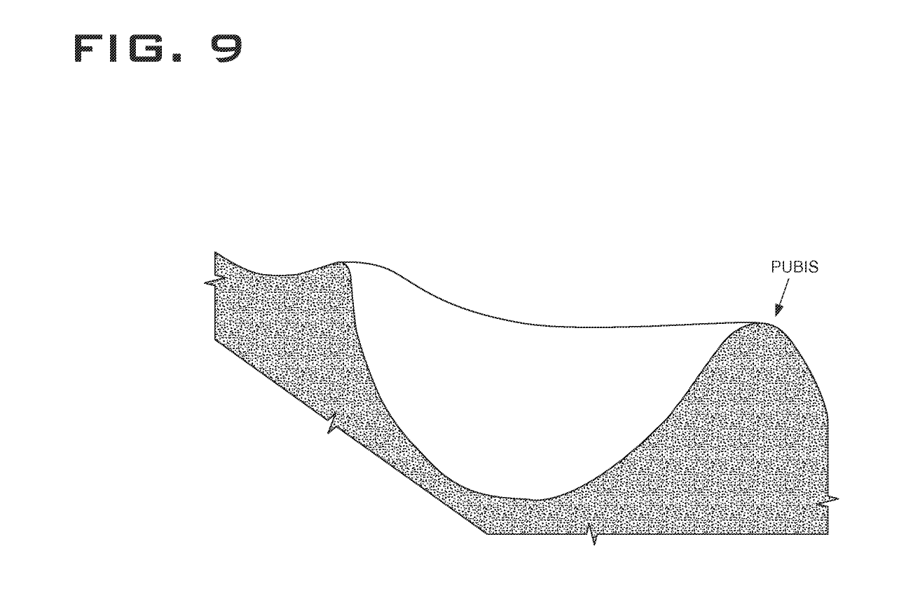

A portion of a pelvis is illustrated in FIG. 1. The pelvis is rotated such that an acetabulum is located near the center of FIG. 1. The ischium, pubis, and ilium bone portions within the acetabulum are labeled in FIG. 1. Cross-section 2 is taken through a portion of the ischium and through the contralateral side of the acetabulum from the ischium and is more particularly illustrated in FIG. 2. Cross-section 7 is taken through a portion of the inferior column and through the contralateral side of the acetabulum from the inferior column and is more particularly illustrated in FIG. 7. Cross-section 9 is taken through a portion of the pubis and through the contralateral side of the acetabulum from the pubis and is more particularly illustrated in FIG. 9.

The following disclosure provides systems, devices, and methods for aligning and implanting acetabular implants (e.g., a shell, liner, cup, cage, augment, etc.) using any suitable alignment tool, or in certain embodiments, preparing an acetabulum to receive an implant using a reamer or other suitable preparation tool. Instruments may include at least one position indicator with a PM surface feature or position indicator that contacts the pelvis within the acetabulum and provides a predetermined orientation of the implant (or in certain embodiments, the preparation device) relative to a patient's anatomy. The systems, devices, and methods may further include a translational feature which permits unilateral translation along a shaft of the alignment tool or preparation tool, and in certain embodiments, includes an orientation feature that positions an acetabular component (or preparation tool) to that of the guide in a particular orientation.

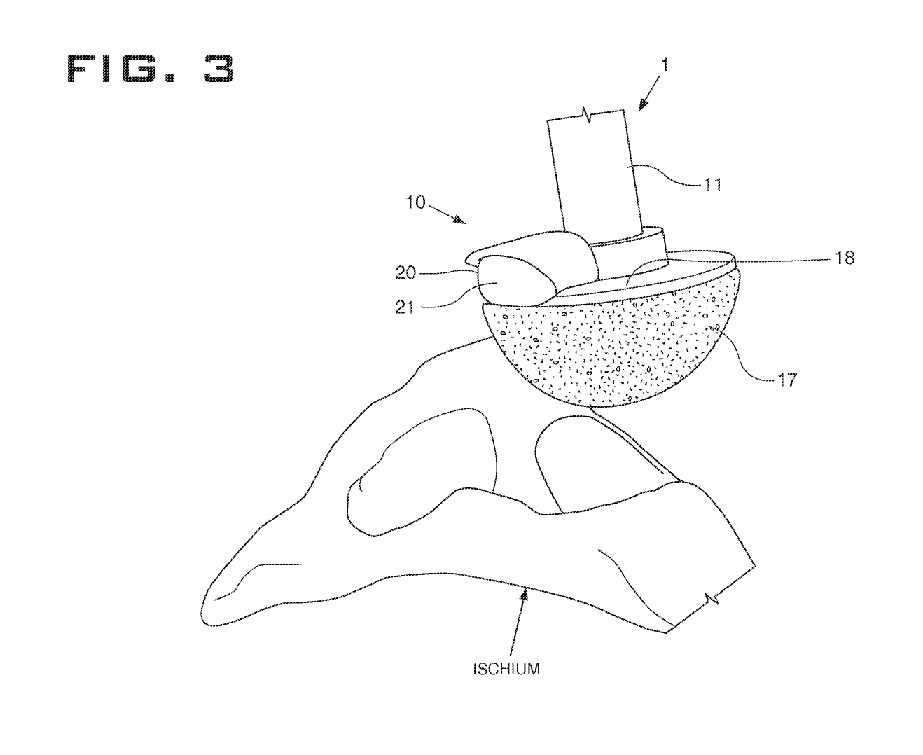

Portions of a prosthetic hip system 1 being directed toward an acetabulum of a pelvis are shown in FIG. 3. The prosthetic hip system 1 depicted includes an acetabular component alignment guide 10. The illustrated acetabular component alignment guide 10 is configured to match a part of the ischium within the acetabulum, as will be more particularly described below. The alignment guide described in this embodiment is the acetabular component alignment guide 10; however, in other embodiments, an alignment guide may appropriately position a reamer or other suitable preparation tool by use of positioned indicators that are employed similarly with the instruments and position indicators disclosed in association with the acetabular component alignment guide 10.

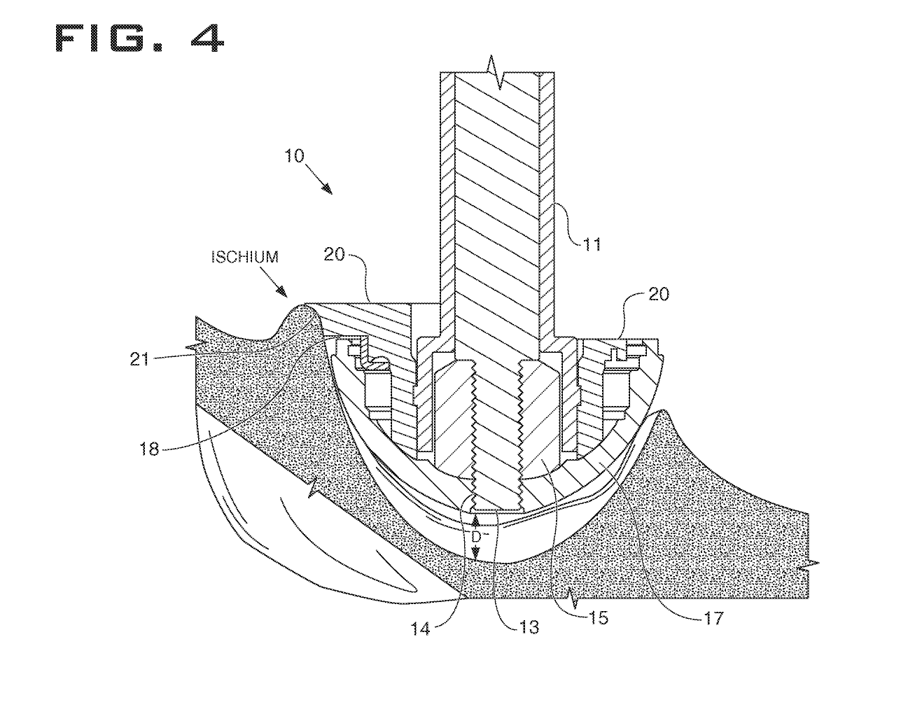

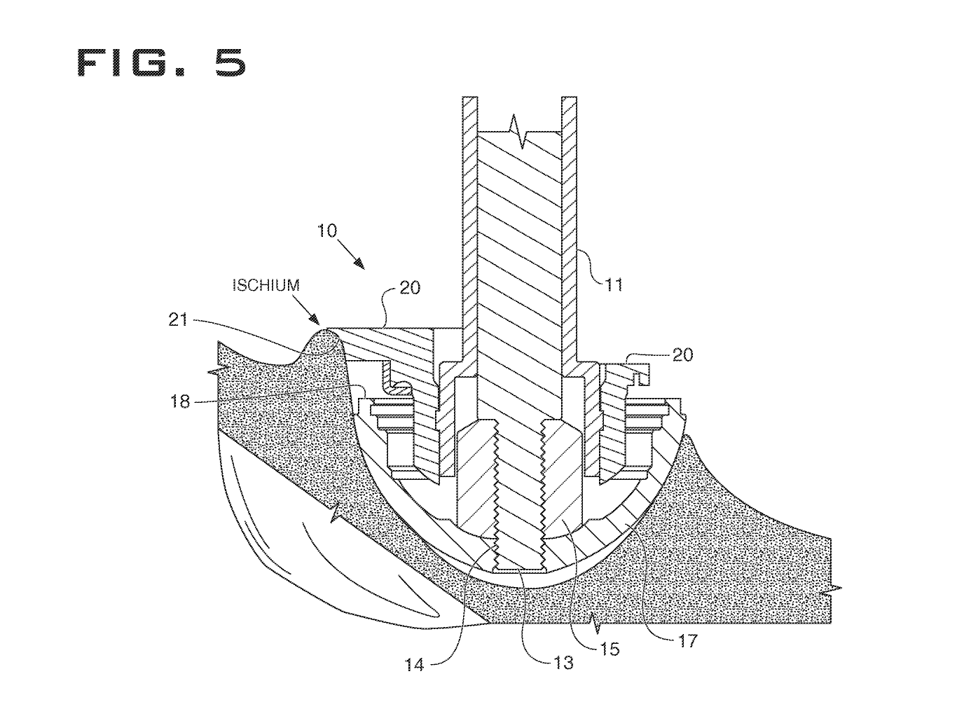

An embodiment of the acetabular component alignment guide 10 is illustrated in FIGS. 3-5. The illustrated acetabular component alignment guide 10 includes a sleeve 11, a shaft 13, an attachment 15, and an insertion guide 20. The shaft 13 shown has a distal threaded portion 14 that is shown in FIGS. 4 and 5 coupled through the attachment 15 and to an acetabular component 17. The attachment 15 may be coupled to the shaft 13 to better distribute an impaction load applied to the shaft 13. Furthermore, the attachment 15 may be configured to more closely match the distal end diameter of the sleeve 11 to better center and guide the sleeve 11 during translation. In certain embodiments, the sleeve 11 may not have an increased distal diameter and there may be no need for the attachment 15 to assist with centering or guiding the sleeve 11 during translation, in which case the attachment 15 may not be included. In some cases, the sleeve 11 and a shaft 13 may have complementary diameters along substantially all of their lengths, which would at least in some regards obviate the need for an attachment such as the attachment 15. The acetabular component 17 illustrated includes a top rim surface 18. The top rim surface 18 may be a substantially continuous planar surface, substantially continuous around a circumference, or may include one or more ridges, bumps, indentations, cutouts, or other discontinuities.

The insertion guide 20 illustrated in FIGS. 3-5 includes a position indicator 21 that is configured to match with a portion of a patient's ischium to orient the insertion guide 20, and consequently the acetabular component 17 to which the insertion guide 20 is coupled, relative to the patient's acetabulum. In this example, alignment with a portion of the patient's ischium is preplanned. In other embodiments, a preplanned anatomical structure may be any structure that may be determined preoperatively and is accessible during a procedure. Anatomical structures may be determined preoperatively by establishing size and shape characteristics of the anatomical structures. For example, size and shape characteristics may be obtained with an imaging device. MRI scans or CT scans may be used to automatically obtain three-dimensional models of a patient's anatomy. Alternatively, two-dimensional imaging devices, such as a radiograph, may be used from more than one angle to approximate a three-dimensional model that may provide adequate size and shape characteristics. Any other type of imaging device capable of determining size and shape characteristics of patients from which a patient-matched instrument may be derived is also contemplated for use in defining a preplanned anatomical structure.

The acetabular component alignment guide 10 depicted has a first section embodied at least in the shaft 13 that is connectable to the acetabular component 17. The shaft 13 includes the distal threaded portion 14 by which the shaft 13 is connected to the acetabular component 17. In other embodiments, a shaft and an acetabular component may be connected by a snap fit connection, by an interference fit, by abutment, or by a connection of any effective type. In the illustrated embodiment, the first section is shown connected to a concave portion of the acetabular component 17. In other embodiments, a first section may be connected to an acetabular component by passing through or around the concave portion of the acetabular component and connecting to the convex portion of the acetabular component.

A second section of the acetabular component alignment guide 10 may be coupled to the first section and is configured to contact a top rim surface of the acetabular component. For example, as illustrated in FIGS. 3 and 4, the shaft 13 through the sleeve 11 is coupled to the insertion guide 20, which contacts the top rim surface 18 of the acetabular component 17. This contact provides a definitive positional relationship of the shaft 13 relative to the insertion guide 20, particularly with reference to the dimension of possible translation of the insertion guide 20 relative to the longitudinal axis of the shaft 13. The contact provided in some embodiments is direct contact between a portion of the second section and a top rim surface of an acetabular component that does not include any other intervening parts. By such direct contact, positional control between a second section and an acetabular component is not dependent on connections with other components. Some embodiments may not include components in contact with a top rim surface of an acetabular component.

In the illustrated embodiment, the second section includes a portion of the insertion guide 20, but not the whole of the insertion guide 20. In particular, the second section includes at least the portion of the insertion guide 20 that contacts the top rim surface 18 of the acetabular component 17. In this embodiment, the portion of the insertion guide 20 that extends beyond the diameter of the acetabular component and is configured to match with a preplanned anatomical structure of a patient is an alignment structure. In the illustrated embodiment, the term diameter is applied to the acetabular component 17 as viewed from above, which is a round projection. The term diameter may also be used for shapes that are not precisely round by reference to the general outer extents of a shape, whether round, oval, rectangular, or having combinations of many sides or curvatures. The alignment structure depicted is coupled to the second section and is configured to orient the acetabular component alignment guide 10 relative to the patient's acetabulum. The alignment structure of the insertion guide 20 includes a position indicator surface 21 that has been preplanned to contact at least a part of the patient's ischium inside the acetabulum. The position indicator is also flush with the top of the patient's ischium in the illustrated embodiment, which provides an additional positional control. In some embodiments, in addition to the alignment structure being configured to match with a preplanned anatomical structure, an acetabular component or other instrument that is coupled directly or indirectly with the alignment structure may also be configured to contact a preplanned anatomical structure such that the alignment structure and the acetabular component or other instrument work in combination to achieve component alignment.

As illustrated by the progression of the device from FIG. 4 to FIG. 5, some embodiments of the acetabular component alignment guide are configured to enable the first section to translate toward the patient's acetabulum relative to the alignment structure. A translation distance D is shown in FIG. 4, but is merely an example. In other embodiments and for other types of devices, the distance of translation may be greater or smaller relative to the size of the device. Where the shaft 13 is considered a part of the first section, and the sleeve 11 is considered a part of the second section, the shaft 13 slides within the sleeve when the first section is translated toward the patient's acetabulum, which is fixed relative to the second section by the coupling of the second section to the alignment structure and the fixed location of the alignment structure relative to the position indicator surface 21 that is retained in contact with of the patient's ischium, as shown in FIG. 5. In some embodiments, the first section may be keyed to the second section such that movement toward the patient's acetabulum is permitted, but rotational movement about the axis of movement toward the patient's acetabulum is restricted or eliminated. For example, the shaft 13 may be keyed to the sleeve 11 such that movement toward the patient's acetabulum, as illustrated in the progression of FIG. 4 to FIG. 5, is permitted, but rotational movement about the axis of the shaft 13 is restricted or eliminated. This functionality may be accomplished, for example and without limitation, with a longitudinal notch or slot in one of the sleeve 11 or the shaft 13 that is mated with a protrusion from the other of the sleeve 11 or shaft 13.

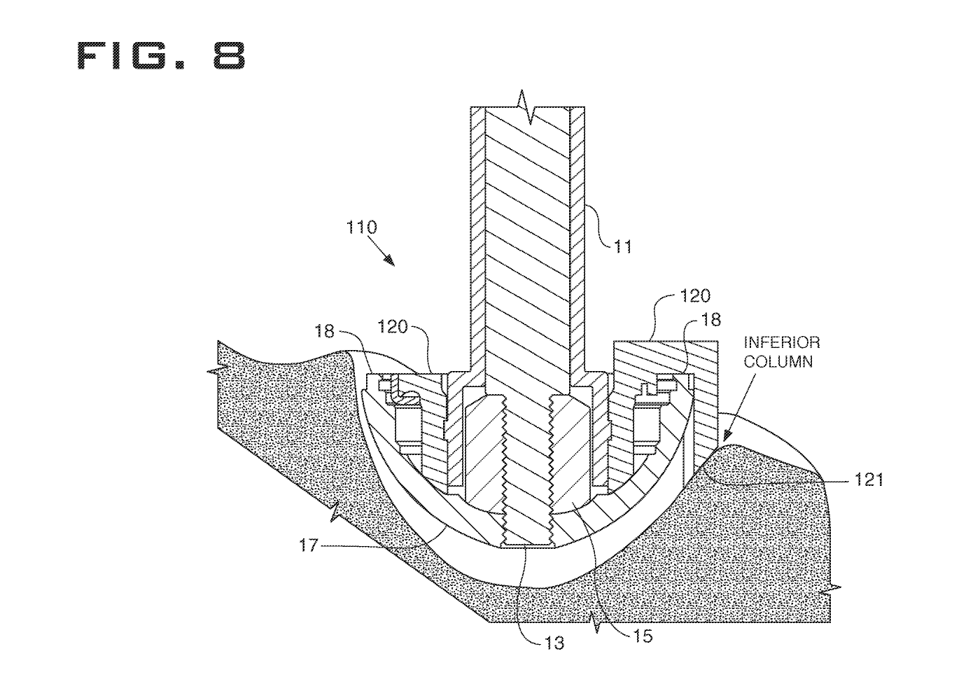

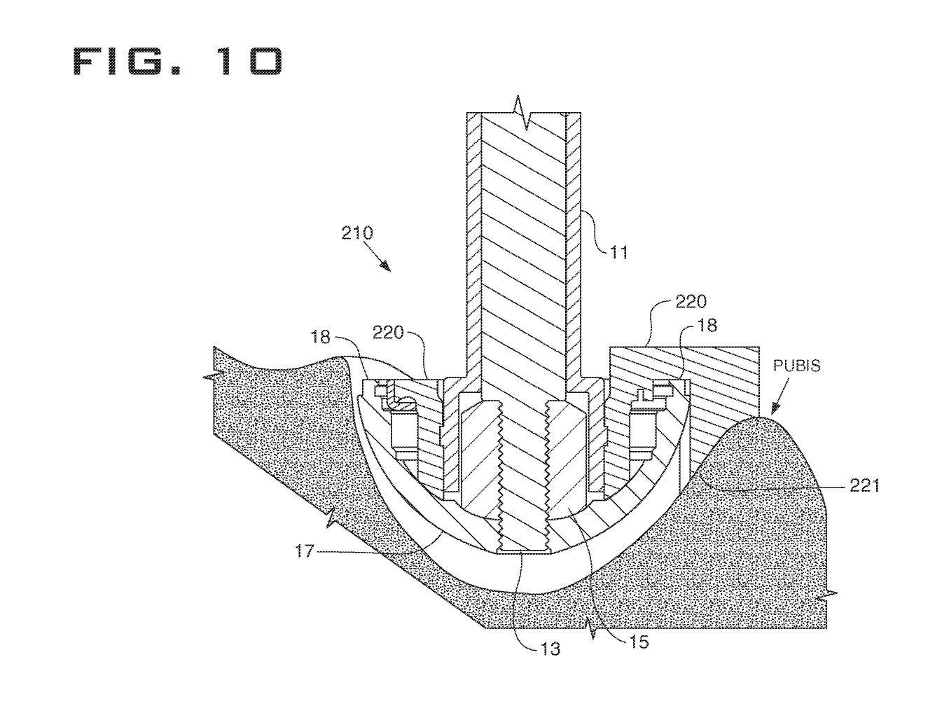

The illustrated position indicator surface 21 of the alignment structure of the insertion guide 20 is preplanned to contact at least a part of the patient's ischium inside the acetabulum. In various embodiments of the invention, a position indicator surface may be preplanned to contact or match any point or surface on an anatomical structure within a patient's acetabulum. Some embodiments, such as the embodiment illustrated in FIGS. 3-5, provide for a preplanned anatomical structure to be matched that is at least a part of the patient's ischium inside the acetabulum and is above the rim of the acetabular component 18 when the acetabular component 17 is seated in the patient's acetabulum. Other embodiments, such as the embodiments illustrated in FIGS. 8 and 10, provide for a preplanned anatomical structure to be matched that is at least a part of the patient's inferior column region of the pelvis and pubis, respectively.

The embodiment shown in FIG. 8 includes an acetabular component alignment guide 110. The illustrated acetabular component alignment guide 110 includes a sleeve 11, a shaft 13, an attachment 15, and an insertion guide 120. The shaft 13 shown has a distal threaded portion 14 that is shown in FIGS. 4 and 5 coupled through the attachment 15 and to an acetabular component 17. The attachment 15 may be coupled to the shaft 13 to better distribute an impaction load applied to the shaft 13. Furthermore, the attachment 15 may be configured to more closely match the distal end diameter of the sleeve 11 to better center and guide the sleeve 11 during translation. In certain embodiments, the sleeve 11 may not have an increased distal diameter and there may be no need for the attachment 15 to assist with centering or guiding the sleeve 11 during translation, in which case the attachment 15 may not be included. In some cases, the sleeve 11 and a shaft 13 may have complementary diameters along substantially all of their lengths, which would at least in some regards obviate the need for an attachment such as the attachment 15. The acetabular component 17 illustrated includes a top rim surface 18. The top rim surface 18 may be a substantially continuous planar surface, substantially continuous around a circumference, or may include one or more ridges, bumps, indentations, cutouts, or other discontinuities.

The insertion guide 120 illustrated in FIG. 8 includes a position indicator 121 that is configured to match with a portion of a patient's pelvic inferior column region to orient the insertion guide 120, and consequently the acetabular component 17 to which the insertion guide 120 is coupled, relative to the patient's acetabulum. In this example, alignment with a portion of the patient's pelvic inferior column region was preplanned. In other embodiments, a preplanned anatomical structure may be any structure that may be determined preoperatively and is accessible during a procedure.

The acetabular component alignment guide 110 depicted has a first section embodied at least in the shaft 13 that is connectable to the acetabular component 17. The shaft 13 includes the distal threaded portion 14 by which the shaft 13 is connected to the acetabular component 17. In other embodiments, a shaft and an acetabular component may be connected by a snap fit connection, by an interference fit, by abutment, or by a connection of any effective type. In the illustrated embodiment, the first section is shown connected to a concave portion of the acetabular component 17. In other embodiments, a first section may be connected to an acetabular component by passing through or around the concave portion of the acetabular component and connecting to the convex portion of the acetabular component.

A second section of the acetabular component alignment guide 110 may be coupled to the first section and is configured to contact a top rim surface 18 of the acetabular component. For example, as illustrated in FIG. 8, the shaft 13 through the sleeve 11 is coupled to the insertion guide 120, which contacts the top rim surface 18 of the acetabular component 17. This contact provides a definitive positional relationship of the shaft 13 relative to the insertion guide 120, particularly with reference to the dimension of possible translation of the insertion guide 120 relative to the longitudinal axis of the shaft 13. The contact provided in some embodiments is direct contact between a portion of the second section and a top rim surface of an acetabular component that does not include any other intervening parts. By such direct contact, positional control between a second section and an acetabular component is not dependent on connections with other components. Some embodiments may not include components in contact with a top rim surface of an acetabular component.

In the illustrated embodiment, the second section includes a portion of the insertion guide 120, but not the whole of the insertion guide 120. In particular, the second section includes at least the portion of the insertion guide 120 that contacts the top rim surface 18 of the acetabular component 17. In this embodiment, the portion of the insertion guide 120 that extends beyond the diameter of the acetabular component and is configured to match with a preplanned anatomical structure of a patient is an alignment structure. In the illustrated embodiment, the term diameter is applied to the acetabular component 17 as viewed from above, which is a round projection. The term diameter may also be used for shapes that are not precisely round by reference to the general outer extents of a shape, whether round, oval, rectangular, or having combinations of many sides or curvatures. The alignment structure depicted is coupled to the second section and is configured to orient the acetabular component alignment guide 110 relative to the patient's acetabulum. The alignment structure of the insertion guide 120 includes a position indicator surface 121 that has been preplanned to contact at least a part of the patient's pelvic inferior column region inside the acetabulum. The position indicator of some embodiments may extend throughout or near the acetabulum as far as may be practical to provide increased contact area so long as access to the implantation area of the device to be implanted is not impeded. In some embodiments, in addition to the alignment structure being configured to match with a preplanned anatomical structure, an acetabular component or other instrument that is coupled directly or indirectly with the alignment structure may also be configured to contact a preplanned anatomical structure such that the alignment structure and the acetabular component or other instrument work in combination to achieve component alignment.

Similar to the translation illustrated in FIGS. 4 and 5, the acetabular component alignment guide 120 illustrated in FIG. 8 is configured to enable the first section to translate toward the patient's acetabulum relative to the alignment structure 120. Where the shaft 13 is considered a part of the first section and the sleeve 11 is considered a part of the second section, the shaft 13 slides within the sleeve when the first section is translated toward the patient's acetabulum, which is fixed relative to the second section by the coupling of the second section to the alignment structure and the fixed location of the alignment structure relative to the position indicator surface 121 that is retained in contact with of the patient's pelvic inferior column region. In some embodiments, the first section may be keyed to the second section such that movement toward the patient's acetabulum is permitted, but rotational movement about the axis of movement toward the patient's acetabulum is restricted or eliminated. For example, the shaft 13 may be keyed to the sleeve 11 such that movement toward the patient's acetabulum is permitted but rotational movement about the axis of the shaft 13 is restricted or eliminated. This functionality may be accomplished, for example and without limitation, with a longitudinal notch or slot in one of the sleeve 11 or the shaft 13 that is mated with a protrusion from the other of the sleeve 11 or shaft 13.

The embodiment shown in FIG. 10 includes an acetabular component alignment guide 210. The illustrated acetabular component alignment guide 210 includes a sleeve 11, a shaft 13, an attachment 15, and an insertion guide 220. The shaft 13 shown has a distal threaded portion 14 that is shown in FIGS. 4 and 5 coupled through the attachment 15 and to an acetabular component 17. The attachment 15 may be coupled to the shaft 13 to better distribute an impaction load applied to the shaft 13. Furthermore, the attachment 15 may be configured to more closely match the distal end diameter of the sleeve 11 to better center and guide the sleeve 11 during translation. In certain embodiments, the sleeve 11 may not have an increased distal diameter and there may be no need for the attachment 15 to assist with centering or guiding the sleeve 11 during translation, in which case the attachment 15 may not be included. In some cases, the sleeve 11 and a shaft 13 may have complementary diameters along substantially all of their lengths, which would at least in some regards obviate the need for an attachment such as the attachment 15. The acetabular component 17 illustrated includes a top rim surface 18. The top rim surface 18 may be a substantially continuous planar surface, substantially continuous around a circumference, or may include one or more ridges, bumps, indentations, cutouts, or other discontinuities.

The insertion guide 220 illustrated in FIG. 10 includes a position indicator 221 that is configured to match with a portion of a patient's pelvic inferior column region to orient the insertion guide 220, and consequently the acetabular component 17 to which the insertion guide 220 is coupled, relative to the patient's acetabulum. In this example, alignment with a portion of the patient's pubis was preplanned. In other embodiments, a preplanned anatomical structure may be any structure that may be determined preoperatively and is accessible during a procedure.

The acetabular component alignment guide 210 depicted has a first section embodied at least in the shaft 13 that is connectable to the acetabular component 17. The shaft 13 includes the distal threaded portion 14 by which the shaft 13 is connected to the acetabular component 17. In other embodiments, a shaft and an acetabular component may be connected by a snap fit connection, by an interference fit, by abutment, or by a connection of any effective type. In the illustrated embodiment, the first section is shown connected to a concave portion of the acetabular component 17. In other embodiments, a first section may be connected to an acetabular component by passing through or around the concave portion of the acetabular component and connecting to the convex portion of the acetabular component.

A second section of the acetabular component alignment guide 210 may be coupled to the first section and is configured to contact a top rim surface 18 of the acetabular component. For example, as illustrated in FIG. 10, the shaft 13 through the sleeve 11 is coupled to the insertion guide 220, which contacts the top rim surface 18 of the acetabular component 17. This contact provides a definitive positional relationship of the shaft 13 relative to the insertion guide 220, particularly with reference to the dimension of possible translation of the insertion guide 220 relative to the longitudinal axis of the shaft 13. The contact provided in some embodiments is direct contact between a portion of the second section and a top rim surface of an acetabular component that does not include any other intervening parts. By such direct contact, positional control between a second section and an acetabular component is not dependent on connections with other components. Some embodiments may not include components in contact with a top rim surface of an acetabular component.

In the illustrated embodiment, the second section includes a portion of the insertion guide 220 but not the whole of the insertion guide 220. In particular, the second section includes at least the portion of the insertion guide 220 that contacts the top rim surface 18 of the acetabular component 17. In this embodiment, the portion of the insertion guide 220 that extends beyond the diameter of the acetabular component and is configured to match with a preplanned anatomical structure of a patient is an alignment structure. In the illustrated embodiment, the term diameter is applied to the acetabular component 17 as viewed from above, which is a round projection. The term diameter also may be used for shapes that are not precisely round by reference to the general outer extents of a shape, whether round, oval, rectangular, or having combinations of many sides or curvatures. The alignment structure depicted is coupled to the second section and is configured to orient the acetabular component alignment guide 210 relative to the patient's acetabulum. The alignment structure of the insertion guide 220 includes a position indicator surface 221 that has been preplanned to contact at least a part of the patient's pubis inside the acetabulum. The position indicator of some embodiments may extend throughout or near the acetabulum as far as may be practical to provide increased contact area so long as access to the implantation area of the device to be implanted is not impeded. In some embodiments, in addition to the alignment structure being configured to match with a preplanned anatomical structure, an acetabular component or other instrument that is coupled directly or indirectly with the alignment structure may also be configured to contact a preplanned anatomical structure such that the alignment structure and the acetabular component or other instrument work in combination to achieve component alignment.

Similar to the translation illustrated in FIGS. 4 and 5, the acetabular component alignment guide 220 illustrated in FIG. 10 is configured to enable the first section to translate toward the patient's acetabulum relative to the alignment structure 220. Where the shaft 13 is considered a part of the first section and the sleeve 11 is considered a part of the second section, the shaft 13 slides within the sleeve when the first section is translated toward the patient's acetabulum, which is fixed relative to the second section by the coupling of the second section to the alignment structure and the fixed location of the alignment structure relative to the position indicator surface 221 that is retained in contact with of the patient's pelvic inferior column region. In some embodiments, the first section may be keyed to the second section such that movement toward the patient's acetabulum is permitted but rotational movement about the axis of movement toward the patient's acetabulum is restricted or eliminated. For example, the shaft 13 may be keyed to the sleeve 11 such that movement toward the patient's acetabulum is permitted, but rotational movement about the axis of the shaft 13 is restricted or eliminated. This functionality may be accomplished, for example and without limitation, with a longitudinal notch or slot in one of the sleeve 11 or the shaft 13 that is mated with a protrusion from the other of the sleeve 11 or shaft 13.

Other embodiments of an acetabular component alignment guide may include a first section that is connectable to an acetabular component and an alignment structure that is coupled to the first section and extends beyond the diameter of the acetabular component and is configured to match at least a part of a patient's ischium inside the acetabulum to orient the acetabular component alignment guide relative to the patient's acetabulum. Certain embodiments do not necessarily have one or more sections that contact a top rim surface of the acetabular component. Other features of such embodiments may be essentially similar to the embodiments described in association with FIGS. 3-5.

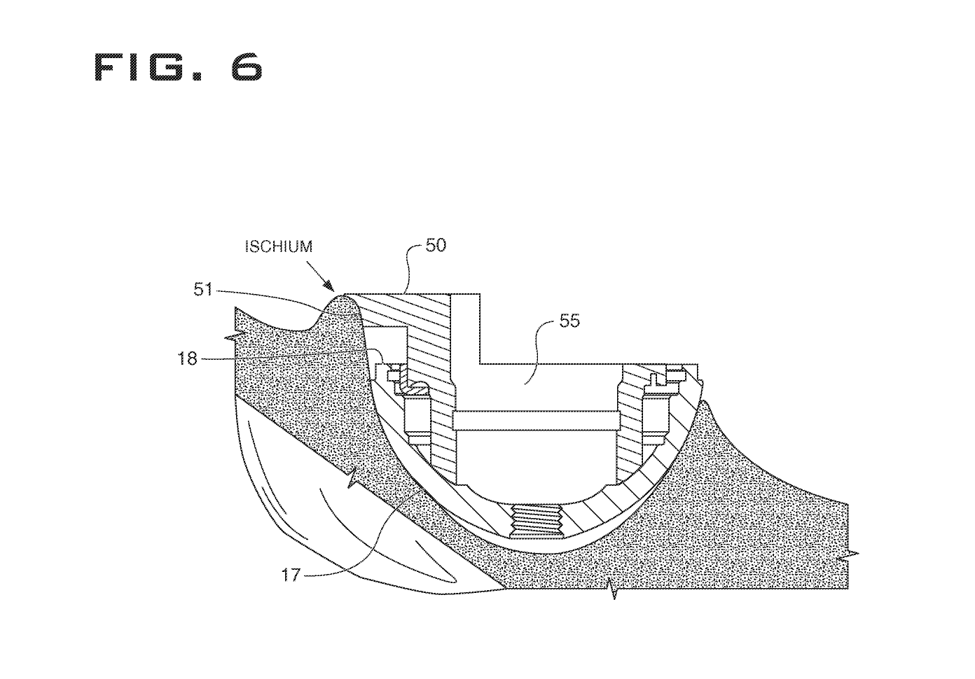

In some embodiments, a fixation guide may be used in addition to an insertion guide. In the illustrated embodiments for example, once an insertion guide such as the insertion guides 20, 120, 220 (FIGS. 3-5, 8, 10) have been removed, a fixation guide such as fixation guide 50 (FIG. 6) may be used to further prepare for or implant a device. A fixation guide may include various temporary fixation structures, and in certain embodiments the fixation structures used in the insertion guide may be found in the fixation guide. Using similar features allows for interchangeability between a given implant and the series of guides. A fixation guide may also include position indicators and alignment structures that match patient anatomy. The fixation guide 50 includes a position indicator 51 that is configured to match with a portion of a patient's ischium to orient the fixation guide 50, and consequently the acetabular component 17 to which the fixation guide 50 is coupled, relative to the patient's acetabulum. The position indicators and alignment structures can reference the same or different parts of the patient anatomy referenced by the insertion guide; however, the placement of an implant with respect to the patient's anatomical reference frame would be the same. The position indicators and alignment structures of the fixation guide are generally shorter in length than those of the insertion guide because the implant has been seated. For example, the length of fixation guide 50 in FIG. 6 is greater than the length of insertion guide 20 in FIG. 5. The approximate length difference in the fixation guide 50 and the insertion guide 20 is the translation distance D shown in FIG. 4.

In certain embodiments, an additional guide is provided for preparing the implantation site (e.g., the acetabulum). In such cases, the guide for preparation, for example reaming, is the first guide used of the series of guides, with the insertion guide being used second and the fixation guide being the third guide used. The fixation guide 50 is illustrated with a void 55 in its interior. This void may be filled with any device that would be useful to prepare or fix an instrument or implant. For example and without limitation, a drill guide, shaft, drill, cutting or reaming component, or any other device may be placed in or passed through the void and guidance structures placed in the void to assist with the preparation or fixation of an instrument or implant.

Another embodiment of the invention is a prosthetic hip system having a femoral stem configured to couple with a femur of a patient, a femoral head coupled to the femoral stem, an acetabular component configured to couple with the femoral head and configured to couple with an acetabulum of the patient, and an acetabular component alignment guide. The femoral stem and head and the acetabular component may be any type conventionally in use currently or developed and put in use at a later date for which alignment is necessary or desirable. Embodiments of the acetabular component alignment guide of the embodiments include a first section that is connectable to the acetabular component, and an alignment structure that is coupled to the first section and extends beyond the diameter of the acetabular component. The alignment structure may be configured to match at least a part of the patient's ischium inside the acetabulum to orient the acetabular component alignment guide relative to the patient's acetabulum. The acetabular component alignment guide of these embodiments may be of any type described or referenced herein.

Various embodiments of components described herein wholly or their parts individually may be made from any biocompatible material. For example and without limitation, biocompatible materials may include in whole or in part: non-reinforced polymers, reinforced polymers, metals, ceramics and combinations of these materials. Reinforcing of polymers may be accomplished with carbon, metal, or glass or any other effective material. Examples of biocompatible polymer materials include polyamide base resins, polyethylene, low density polyethylene, polymethylmethacrylate (PMMA), polyetheretherketone (PEEK), polyetherketoneketone (PEKK), a polymeric hydroxyethylmethacrylate (PHEMA), and polyurethane, any of which may be reinforced. Example biocompatible metals include stainless steel and other steel alloys, cobalt chrome alloys, tantalum, titanium, titanium alloys, titanium-nickel alloys such as Nitinol and other superelastic or shape-memory metal alloys. Components described herein may be formed by conventional milling or casting processes, by any type of three-dimensional printing or deposition based processes, or by any effective process.

An embodiment of the invention is a method of providing instruments to align a prosthetic hip system. The embodiment may include receiving data about a patient's anatomy, using the received data to establish a three dimensional model of the patient's anatomy, and forming an acetabular component alignment guide. Patient anatomical data received may be from collected specifically for the purpose of forming a guide device or may be data gathered for another purpose. Patient anatomical data may be obtained with an imaging device. MRI scans or CT scans may be used to automatically obtain three-dimensional models of a patient's anatomy. Alternatively, two-dimensional imaging devices, such as a radiograph, may be used from more than one angle to approximate a three-dimensional model that may provide adequate size and shape characteristics.

The acetabular component guide may include at least one surface that interfaces with a surface within the patient's acetabulum such that when the at least one surface is positioned on the patient's anatomy, the acetabular component alignment guide is configured to direct an acetabular component toward a predetermined position in the patient's acetabulum. Acetabular component guides with surfaces that interface with surfaces within the patient's acetabulum are illustrated in FIGS. 3-5 (position indicator 21), FIG. 8 (position indicator 121), and FIG. 10 (position indicator 221).

In some embodiments, the act of forming an acetabular component alignment guide includes forming an acetabular component alignment guide with at least one surface that interfaces with a surface on at least a part of a patient's ischium above the rim of the acetabular component when the acetabular component is seated in the patient's acetabulum. For example and without limitation, the acetabular component alignment guide 20 illustrated in FIGS. 3-5 includes the position indicator 21 that interfaces with a surface on the illustrated ischium above the rim of the acetabular component 17 when the acetabular component is seated in the patient's acetabulum (FIG. 5).

The act of forming an acetabular component alignment guide may include forming an acetabular component alignment guide configured to permit a portion of the acetabular component alignment guide coupled to the acetabular component to translate relative to the at least one surface of the acetabular component alignment guide to direct the acetabular component toward a predetermined position in the patient's acetabulum. Nonlimiting examples of such formed guides are illustrated in FIGS. 3-5, 8, and 10 the translations of which are described in more detail herein.

The act of forming an acetabular component alignment guide may include providing a fixation guide with a least one surface that interfaces with the surface within the patient's acetabulum when the acetabular component is in the predetermined position in the patient's acetabulum. Some such fixation guides may also include one or more guide holes for the placement of fasteners such that the one or more guide holes provide a trajectory into one or more suitable locations in the patient's anatomy, as determined from the data received about the patient's anatomy. Examples of such fixation guides are described and referenced in association with FIG. 6 herein.

Terms such as distal, unilateral, contralateral, above, inside, and the like have been used relatively herein. However, such terms are not limited to specific coordinate orientations but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the invention should not be limited by any of the above-described exemplary embodiments, but should be defined only in accordance with the claims and their equivalents.

What is claimed is:

1. An acetabular component alignment guide comprising:
a first section that is connectable to an acetabular component;
a second section that is coupled to the first section and is configured to contact a top rim surface of the acetabular component; and
an alignment structure that is coupled to the second section and extends beyond the diameter of the acetabular component and is configured to match with a preplanned anatomical structure of a patient to orient the acetabular component alignment guide relative to the patient's acetabulum.

2. The acetabular component alignment guide of claim 1, wherein the first section is connectable to a concave portion of the acetabular component.

3. The acetabular component alignment guide of claim 1, wherein the first section is configured to translate toward the patient's acetabulum relative to the alignment structure.

4. The acetabular component alignment guide of claim 1, wherein the first section includes a shaft and the second section includes a sleeve, wherein the shaft is configured to slide within the sleeve when the first section is translated toward the patient's acetabulum.

5. The acetabular component alignment guide of claim 1, wherein the first section is keyed to the second section such that movement toward the patient's acetabulum is permitted, but rotational movement about the axis of movement toward the patient's acetabulum is restricted.

6. The acetabular component alignment guide of claim 1, wherein the preplanned anatomical structure that the alignment structure is configured to match is an anatomical structure within the patient's acetabulum.

7. The acetabular component alignment guide of claim 1, wherein the preplanned anatomical structure that the alignment structure is configured to match is at least a part of the patient's ischium inside the acetabulum.

8. The acetabular component alignment guide of claim 1, wherein the preplanned anatomical structure that the alignment structure is configured to match is at least a part of the patient's ischium inside the acetabulum and above the rim of the acetabular component when the acetabular component is seated in the patient's acetabulum.

9. The acetabular component alignment guide of claim 1, wherein the preplanned anatomical structure that the alignment structure is configured to match is the patient's inferior column region of the pelvis.

10. The acetabular component alignment guide of claim 1, wherein the preplanned anatomical structure that the alignment structure is configured to match is at least a part of the patient's pubis inside the acetabulum.

11. An acetabular component alignment guide comprising:
a first section that is connectable to an acetabular component; and
an alignment structure that is coupled to the first section and extends beyond the diameter of the acetabular component and is configured to match at least a part of a patient's ischium inside the acetabulum to orient the acetabular component alignment guide relative to the patient's acetabulum.

12. The acetabular component alignment guide of claim 11, wherein the first section is connectable to a concave portion of the acetabular component.

13. The acetabular component alignment guide of claim 11, wherein the first section is configured to translate toward the patient's acetabulum relative to the alignment structure.

14. The acetabular component alignment guide of claim 11, wherein the alignment structure is configured to match at least a part of the patient's ischium above the rim of the acetabular component when the acetabular component is seated in the patient's acetabulum.

15. The acetabular component alignment guide of claim 11, wherein the alignment guide is configured to contact a top rim surface of the acetabular component.

16. A prosthetic hip system comprising:
a femoral stem configured to couple with a femur of a patient;
a femoral head coupled to the femoral stem;
an acetabular component configured to couple with the femoral head and configured to couple with an acetabulum of the patient; and
an acetabular component alignment guide comprising:
a first section that is connectable to the acetabular component, and
an alignment structure that is coupled to the first section and extends beyond the diameter of the acetabular component and is configured to match at least a part of the patient's ischium inside the acetabulum to orient the acetabular component alignment guide relative to the patient's acetabulum.

17. The prosthetic hip system of claim 16, wherein the first section is connectable to a concave portion of the acetabular component.

18. The prosthetic hip system of claim 16, wherein the first section is configured to translate toward the patient's acetabulum relative to the alignment structure.

19. The prosthetic hip system of claim 16, wherein the alignment structure is configured to match at least a part of the patient's ischium above the rim of the acetabular component when the acetabular component is seated in the patient's acetabulum.

20. The prosthetic hip system of claim 16, wherein the alignment guide is configured to contact a top rim surface of the acetabular component.

21. A method of providing instruments to align a prosthetic hip system comprising:
receiving data about a patient's anatomy;
using the received data to establish a three dimensional model of the patient's anatomy; and
forming an acetabular component alignment guide with at least one surface that interfaces with a surface within the patient's acetabulum such that when the at least one surface is positioned on the patient's anatomy, the acetabular component alignment guide is configured to direct an acetabular component toward a predetermined position in the patient's acetabulum.

22. The method of claim 21, wherein the act of forming an acetabular component alignment guide includes forming an acetabular component alignment guide with at least one surface that interfaces with a surface on at least a part of the patient's ischium above the rim of the acetabular component when the acetabular component is seated in the patient's acetabulum.

23. The method of claim 21, wherein the act of forming an acetabular component alignment guide includes forming an acetabular component alignment guide with at least one surface that interfaces with a surface on at least a part of the patient's pubis within the patient's acetabulum.

24. The method of claim 21, wherein the act of forming an acetabular component alignment guide includes forming an acetabular component alignment guide with at least one surface that interfaces with a surface on at least a part of the patient's inferior column within the patient's acetabulum.

25. The method of claim 21, wherein the act of forming an acetabular component alignment guide includes forming an acetabular component alignment guide configured to permit a portion of the acetabular component alignment guide coupled to the acetabular component to translate relative to the at least one surface of the acetabular component alignment guide to direct the acetabular component toward a predetermined position in the patient's acetabulum.

26. The method of claim 21, further comprising providing a fixation guide with a least one surface that interfaces with the surface within the patient's acetabulum when the acetabular component is in the predetermined position in the patient's acetabulum.

27. The method of claim 26, wherein the fixation guide includes one or more guide holes for the placement of fasteners such that the one or more guide holes provide a trajectory into one or more suitable locations in the patient's anatomy, as determined from the data received about the patient's anatomy.
